(19) United States
(12) Patent Application Publication
(10) Pub. No.: US 2010/0227348 A1
(43) Pub. Date: Sep. 9, 2010

(54) METHOD AND DEVICE FOR DETERMINING THE CONCENTRATION OF AN ANALYTE USING FLUORESCENCE MEASUREMENT

(76) Inventors: Wolfgang Petrich, Bad Schonborn (DE); Hans-Peter Haar, Wiesloch (DE); Joachim Hoenes, Zwingenberg (DE); Carina Horn, Biblis (DE)

Correspondence Address: ROCHE DIAGNOSTICS OPERATIONS INC., 9115 Hague Road, Indianapolis, IN, 46250-0457, US

(21) Appl. No.: 12/696,665

(22) Filed: Jan. 29, 2010

Related U.S. Application Data
(63) Continuation of application No. PCT/EP2008/006255, filed on Jul. 30, 2008.

(30) Foreign Application Priority Data
Aug. 1, 2007 (EP) 07015084.2

(51) Int. Cl. C12Q 1/54; C12M 1/34

(52) U.S. Cl. 435/14; 435/287.1

(57) ABSTRACT

A method is presented for determining the concentration of an analyte in a liquid sample using a reagent system. The reaction of the sample with the reagent system results in a change of the quantity of a fluorophore, the quantity of the fluorophore correlating with the concentration of the analyte, a characteristic measurement variable for the quantity of the fluorophore being measured, and the concentration of the analyte being determined using an evaluation algorithm on the basis of the measurement variable. The reagent system is integrated in an analysis system and the fluorescence lifetime of the fluorophore is used in the ascertainment of the concentration of the analyte.

### CLAIM OF PRIORITY

The present application is a continuation application based on and claiming priority to PCT Application No. PCT/EP2008/006255, filed Jul. 30, 2008, which claims the priority benefit of European Application No. 07015084.2, filed Aug. 1, 2007, each of which are hereby incorporated by reference in their entireties.

### TECHNICAL FIELD OF THE INVENTION

The present application relates to a method for determining the concentration of an analyte in a liquid sample, and more particularly to the assaying of medical samples such as blood and/or interstitial liquid, for analytes contained therein, such as glucose.

### BACKGROUND

In methods for determining the concentration of analyte based on a fluorescence measurement, a reagent system is used which reacts with the sample resulting in a change of the amount of a fluorophore. This change of quantity can in principle be both an increase by formation of a fluorophore and also a decrease by consumption of a fluorophore. The reaction system is selected so that the change of the quantity of the fluorophore is characteristic of the desired analytical concentration. The analytical method includes the measurement of a measurement variable which correlates with the quantity of the fluorophore. The concentration is determined on the basis of the resulting measurement values by means of an evaluation algorithm.

Methods of this type are generally known. Reference can be made to the following documents, for example EP 0 293 732 A2, US 2005/0214891 A1, and US 2006/0003397 A1.

All of these publications describe a similar test protocol, in which the reagent system contains the enzyme-coenzyme pair glucose dehydrogenase (GlucDH)/nicotinamide-adenine dinucleotide (NAD). Upon the reaction with this reagent system, under the influence of the GlucDH, a hydride ion is cleaved from the glucose and transferred to the NAD, so that NADH forms. The resulting quantity of NADH is characteristic of the glucose concentration. NADH is a strong fluorophore, whose concentration can be determined by a measurement of the fluorescence intensity. The correlation of the measured fluorescence intensity to the analyte concentration is typically determined by calibration. NADH is also a dye. Therefore, it can be favorable to additionally perform a photometric measurement (for example, measurement of the optical absorption at a specific wavelength).

This type of method has basically been known for some time. For example, reference is made in US 2005/0214891 A1 to a publication of Narayanaswamy et al. from the year 1988, in which a fluorescence measurement using GlucDH and NAD was employed for glucose determination. However, an array of fundamental problems is connected to the measurement of the fluorescence intensity, which are discussed, for example, in the book by J. R. Lakovicz, “Principles of Fluorescence Spectroscopy” (Springer Science and Business Media, 2006), and include the following:
The intensity of the detected fluorescence signal is a function of a plurality of instrument-specific factors, such as the power of the light source, the transmission of the optical components in the light path, or the sensitivity of the detector. Uncontrolled changes of these factors impair the measurement precision.
A further error source in intensity measurements results from nonspecific light, which reaches the detector from the environment and can cause an uncontrolled signal change.
The intensity of the measured fluorescence light is not only a function of the quantity of the fluorophore. Rather it is also significantly influenced by its molecular environment in the sample. In particular processes which are summarized under the term fluorescence quenching contribute thereto in.
The position and orientation of the molecule can change between absorption and emission because in the statistical mean a time in the order of nanoseconds passes between the excitation of a molecule and the emission of a light quantum. Interfering influences result therefrom in regard to the fluorescence intensity, in particular temperature dependence.
The fluorescence is generally excited by ultraviolet light. Photochemical reactions of the electronically excited state may cause bleaching of the fluorophore. This is a further error source.

These problems may be reduced by calibration of the measurement of the fluorescence intensity using a fluorophore whose fluorescence intensity is known. However, this is very complex and is only suitable for sophisticated laboratory measurements.

On this basis, it is an object of the present invention to propose a method which allows with less expense an improved measurement precision, in particular in regard to the described measurement errors and interference. In particular it should be suitable for small devices which operate as simply and cost-effectively as possible.

### SUMMARY

This object and others that will be appreciated by a person of ordinary skill in the art have been achieved according to the embodiments of the present invention disclosed herein. In one embodiment, the present invention comprises a method of the type explained generally above, wherein the reagent system is integrated in an analysis element and the fluorescence lifetime of the fluorophore is used in the determination of the concentration of the analyte. Furthermore the invention refers to an analysis system for performing the method.

It has been established in the context of the invention that in analysis systems in which the reagents of the reagent system are integrated in dry form in an analysis element, the fluorescence lifetime is surprisingly dependent to such a high degree on the analyte concentration that measurement values of this measurement variable may advantageously be used in the algorithm for determining the analytical result (desired concentration). With knowledge of the invention this may be explained as follows for the case of a glucose test using the reagent system GlucDH/NAD.

The NADH formed as a result of the reaction of the glucose with the enzyme-coenzyme pair GlucDH/NAD forms a complex with the GlucDH. This complex formation influences the fluorescence lifetime of the NADH: the fluorescence of the free NADH is short-lived, because molecules in the surrounding liquid act as a quencher. In the complex, the NADH molecule is largely protected from the effect of quenchers and the fluorescence is significantly longer-lived.

These facts are known from the literature. It was, however, completely unexpected that this effect is so pronounced under the conditions prevailing in practical tests that the changes of the analyte concentration in blood samples occurring in practice result in a well-measurable shift of the mean fluorescence lifetime. On the basis of statements in the literature, in particular in document (3), it would be expected that the degree of binding of the complex NADH/GlucDH is very high. The fluorescent light emitted by the complex NADH/GlucDH has a very much higher intensity than the fluorescence of free NADH. Therefore, it would be expected that the observed fluorescence lifetime depends only minimally from the free NADH and the lifetime effect caused by concentration changes in the physiological range is immeasurably low. However, it was established in the context of the invention that under practical conditions a very high proportion of the NADH (over 90%) is not complexed.

The fluorescence lifetime provides a second, independent source of information about the fluorescence of the reaction products, in addition to the fluorescence intensity. It can be used independently (i.e., without other information) for determining the desired analyte concentration. However, it is preferably used in combination with the measurement of the fluorescence intensity. This does not require any additional expense for measurement technology, because typical methods for measuring the fluorescence lifetime simultaneously provide measurement results about the fluorescence intensity.

In order to use the fluorescence lifetime according to the invention for the determination of the desired analyte concentration, it can be expedient to measure it and to introduce the measurement values resulting from the measurement into the evaluation algorithm in any suitable way. However, such a measurement (in the strict meaning, i.e., measurement of previously unknown measurement values) is not mandatory. Rather, the fluorescence lifetime can also be used in a way in which no measurement in the meaning that (previously unknown) measurement values of the fluorescence lifetime are determined, is required. One possibility is to measure the fluorescence intensity within a defined time window, the time window being adapted to the (known) fluorescence lifetime of a fluorophore resulting from the reaction of the sample with the reagent system. The measurement value resulting from such a measurement is related to the fluorescence lifetime. Hereafter it is also designated “lifetime-related fluorescence intensity”. It is used in the evaluation algorithm. Therefore also in this case, it is correct to say that the fluorescence lifetime is used in the evaluation algorithm.

Due to the fact that a measurement of the fluorescence lifetime (in the explained strict meaning) is not absolutely necessary, the analysis instrument of a corresponding analysis system does not necessarily have to have a measuring unit which is adapted for measuring the fluorescence lifetime of the fluorophore. Rather, the possibility alternatively or additionally exists that the measuring unit of the analysis instrument is implemented so that it considers the fluorescence lifetime in another way, for example, using the discussed time window which is set during the intensity measurement.

Suitable methods for measuring the fluorescence lifetime are known. One means that is, in principle, suitable for the invention is a time-resolved fluorescence detection, in which a very short pulse of excitation light is emitted and the curve of the resulting emission curve is detected using suitable detection methods having high time resolution. However, a phase modulation method operating with continuous emission is preferably used in the context of the invention. More detailed information about suitable methods may be taken from the literature. Reference is to be made in particular to the book of Lakovicz cited above, and to the following further publications, as well as the literature cited therein: T. G. Scott et al., “Emission Properties of NADH. Studies of Fluorescence Lifetimes . . . ” (J. Am. Chem. Soc., 1970, 687-695); U.S. Pat. No. 5,485,530; and EP 0 561 653 A1.

Inter alia, the following advantages may be achieved by the present invention:
The problems discussed above are largely avoided. In particular, a calibration of the fluorescence intensity, using a standard fluorophore, is not necessary.
The high-frequency measurement results in a reduction of the influence of interfering ambient light.
The invention allows increased precision, in particular by elimination or reduction of error sources.

The invention is to be explained in more detail by the following figures and examples.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 shows a schematic sketch of the functional components of an analysis system suitable for embodiments of the present invention.

FIG. 2 shows measurement results in regard to the mean lifetime of the fluorescence as a function of the ratio of the concentrations of GlucDH and NADH.

FIG. 3 shows a measurement result in regard to the functional relationship between the glucose concentration and the intensity ratio between short-lived and long-lived fluorescence.

FIG. 4 shows a comparison of the glucose concentration measured according to embodiments of the present invention with the results of a reference measurement method.

In order that the present invention may be more readily understood, reference is made to the following detailed descriptions and examples, which are intended to illustrate the present invention, but not limit the scope thereof.

### DETAILED DESCRIPTION OF EMBODIMENTS OF THE PRESENT INVENTION

The following descriptions of the embodiments are merely exemplary in nature and are in no way intended to limit the present invention or its application or uses.

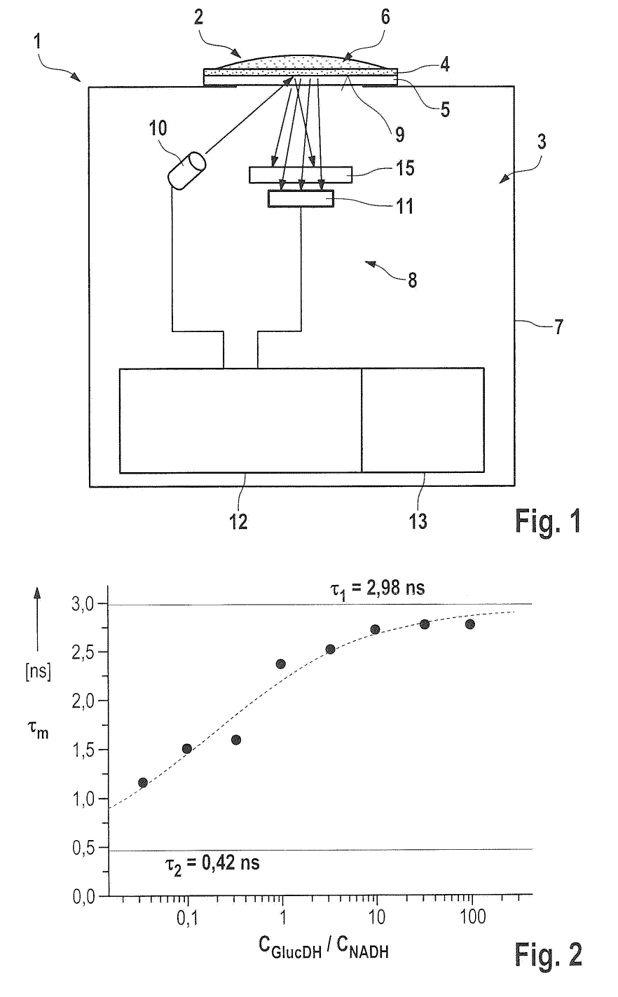

The analysis system shown in very schematic form in FIG. 1 comprises two components adapted to one another, namely an analysis element 2 and an analysis instrument 3. The analysis element 2 comprises a reagent layer 4, which contains a reagent system in a suitable matrix, such as a gel matrix. The reagent layer 4 is located on an optically transparent carrier 5. The reagents of the reaction system are embedded in the matrix of the reagent layer 4 in dry form. When a sample liquid 6 is dispensed onto the free surface of the reagent layer 4 and penetrates therein, the reagents of the reagent system are dissolved and a reaction of the sample liquid occurs, which results in a change of the quantity of a fluorophore, which is a component of the reagent system. Further information about such analysis elements is provided, for example, in the documents U.S. Pat. No. 3,802,842; U.S. Pat. No. 4,061,468; and US 2006/0003397.

The housing 7 of the analysis instrument 3 contains a measuring unit, designated identified as a whole by 8. It is adapted for determining a measurement variable which is characteristic for the quantity of the fluorophore. The measuring unit 8 includes an excitation light source 10, such as a laser or an LED, whose light is directed from the back side (through the optically transparent carrier 5) onto a surface of the reagent layer 4 which faces away from the sample dispensing side and toward the optically transparent carrier 5. Secondary light is emitted from the analysis zone 9 toward a detector 11 and is detected thereby. The resulting measuring signal is supplied to a signal processing unit 12 and amplified, processed, and digitized in a usual manner. The resulting digitized measurement values are supplied to an analysis unit 13 which contains the required software and hardware to determine the desired concentration of the analyte from the digitized measurement values.

The measuring unit 8 is adapted for detecting fluorescent light emitted from the analysis zone 9. For this purpose, the access of the primary light originating from the excitation light source 10 is blocked, using typical means such as a filter element 15 shown in FIG. 1, in such a manner that the fluorescent light, which is emitted at a longer wavelength, can be detected selectively by the detector 11.

So far the design of the analysis system according to the invention is conventionally and therefore does not have to be explained in greater detail. More detailed information is available, for example, from the cited documents of the prior art.

A special feature of the analysis system according to the invention shown in FIG. 1 is that the measuring unit 8 is capable of measuring the fluorescence lifetime and/or to take it into account (in the context of the measurement of another measurement variable). As already noted, the fluorescence lifetime measurement is preferably performed using a phase modulation method. The light emitted from the excitation light source 10 is modulated at high frequency and the desired information about the fluorescence lifetime is determined from a phase shift which is measured by means of the detector 11 and the signal processing unit 12. Again a more detailed explanation in this regard is not necessary, because supplementary information is available from the literature, in particular the above-mentioned documents.

FIG. 2 shows experimental results from the research work on which the invention is based. The mean fluorescence lifetime τₘ is shown for various concentration ratios of a liquid mixture of GlucDH and NADH. An NADH solution having a concentration of 1.2 mg/L was admixed with varying quantities of GlucDH to prepare the concentrations specified on the abscissa. FIG. 2 shows that the shift of the ratio between free and complexed NADH, which is caused by the variation of the concentration of GlucDH, can be observed well by lifetime measurements, the measurement curve approaching the limiting values τ₁=2.98 ns and τ₂=0.42 ns for very high and very low GlucDH concentrations. The fluorescence lifetime is a measurement variable which is characteristic of the quantity of the fluorophore, as shown by the fact that the degree of complex formation is a function of the amount of the fluorescent species.

In the context of the invention it was derived from the measurement results shown in FIG. 2 that the change of the lifetime can be used as information for quantifying an analyte in fluorescence tests. These findings can be used analytically in various ways.

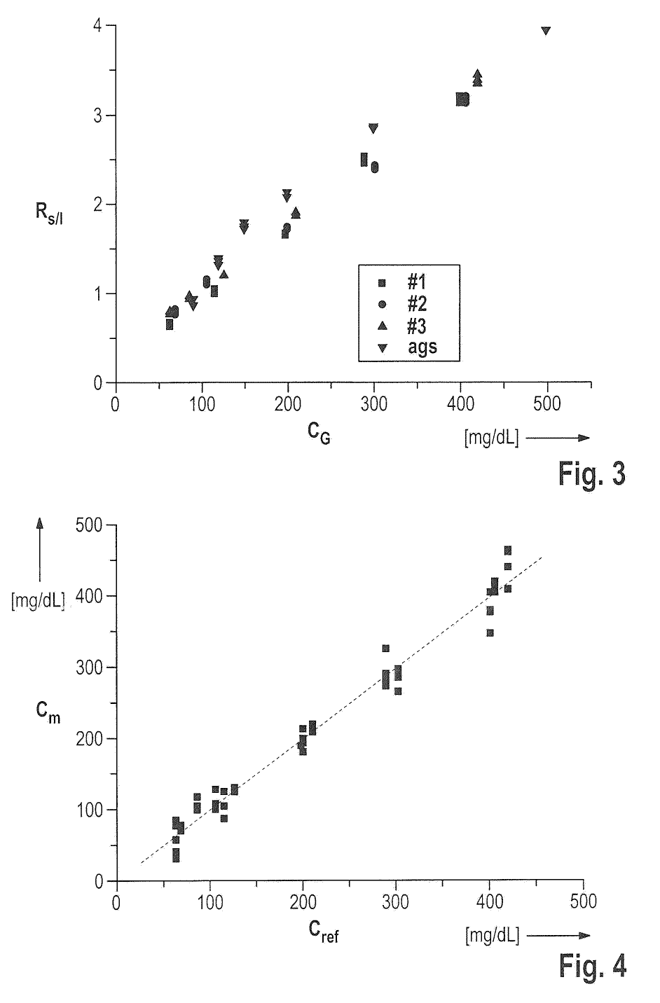

FIG. 3 illustrates a preferred possibility in which the measurement of the fluorescence lifetime is used for determining a lifetime-related fluorescence intensity (LRFI). Such an LRFI value describes the intensity of the fluorescence as a function of the fluorescence lifetime and thus forms a measure of the relationship of the quantity of a plurality of fluorescent species, which are distinguished by different mean lifetimes, such as complexed NADH in proportion to free NADH. An LRFI value can be determined, for example, on the basis of fluorescence signals, which are excited by short light pulses and are measured at high resolution of time. The decay of the fluorescence signal is a logarithmic function, which is a function of the mean fluorescence lifetimes of the fluorescent species and their concentration. The separate intensities of the two signal components may be determined by mathematical fitting of such measurement curves.

FIG. 3 shows the dependence of such LRFI values on the glucose concentration for a glucose fluorescence test using analysis elements as are described in US 2005/0214891 A1 and US 2006/0003397 A1. A ratio R_{s/l} between the LRFI value for short-lived free NADH and the LRFI value for long-lived complexed NADH is shown. The measurements were performed using blood samples of different hematocrit values and using an aqueous glucose solution. The resulting measurement values are identified by different symbols in FIG. 3. Symbols for #1 through #3 indicate the numbers of samples having different hematocrit concentrations (very high—normal range—very low); “ags” means “aqueous glucose solution”. The correlation between the glucose concentration and the R_{s/l} value is surprisingly good, the measured values being largely independent of the hematocrit value, i.e., of the concentration of the blood cells in the sample.

FIG. 3 shows a first example of a preferred embodiment of the invention, in which two LRFI values are related to one another. R_{s/l} was calculated according to

$${R}_{s / 1} = \frac{{A}_{2} \cdot {τ}_{2}}{{A}_{1} \cdot {τ}_{1}}$$

Therein A designates the amplitude, i.e., a measure of the intensity, and τ designates the fluorescence lifetime of the two fluorescent species having different lifetimes. The value R_{s/l} is calibrated against the analyte concentration and then used in the evaluation algorithm as a measure for the analyte concentration.

There are numerous other possibilities for relating LRFI values to one another so that a computed variable is formed, which can be used, via a calibration, as a measure for the desired analyte concentration. For example, a mean fluorescence lifetime τₘ can be calculated according to

$${τ}_{m} = \frac{{A}_{1} {τ}_{1} + {A}_{2} {τ}_{2}}{{A}_{1} + {A}_{2}}$$

According to a particularly preferred embodiment, area-weighted averaging is performed according to

$${τ}_{m} = \frac{{A}_{1} {τ}_{1}^{2} + {A}_{2} {τ}_{2}^{2}}{{A}_{1} {τ}_{1} + {A}_{2} {τ}_{2}}$$

All of these examples share the feature that the fluorescence lifetimes and the fluorescence intensities of two fluorescent species are used as characteristic measurement variables for the analysis. They are related to one another according to a predefined mathematical equation in order to determine a computed variable, which can be calibrated against the desired analyte concentration. The use of two simultaneously measured values and the calculation of a quotient has the result that measurement errors which influence both fluorescence measurement values in the same way, for example, due to contaminants or small flaws in the optical measuring channel, cause no or little deterioration of the measurement result, less than in previously known measurements of the fluorescence intensity.

The measurement of LRFI values is an example of the fact that in the context of the invention, a measurement of a fluorescence lifetime (in the strict meaning) is not absolutely necessary. As described above, the measuring unit of the analysis instrument can also be implemented so that it detects a fluorescence intensity of a predefined time window and an LRFI value is thus measured. Preferably two or more LRFI values of this type are related to one another in order to determine a computed variable which can be calibrated against the analyte concentration, similarly as explained in the above examples.

FIG. 4 shows the correlation of glucose concentration values Cₘ, which were determined according to the invention, with glucose concentration values C_{ref}, which were determined using a reference method. Again, it is shown that the analysis values determined on the basis of the fluorescence lifetime correspond astoundingly well with the values of the reference method.

The invention is not only applicable for the described system, in which glucose is determined with the aid of GlucDH and NAD. Rather, it may be applied very generally for cases in which a fluorophore is used which fluoresces in at least two species having different fluorescence lifetimes. In particular, the invention is suitable for tests in which the reaction of the sample with the reagent system includes complex formation with participation of at least two molecules, the fluorophore being one of the at least two molecules. The molecules which participate in the complex formation are preferably an enzyme/coenzyme pair, the coenzyme preferably being the fluorophore.

The fluorophore is preferably a coenzyme, selected from the group comprising NADH/H⁺ and NADPH/H⁺, including their derivatives. In particular, derivatives are suitable in which the molecule part essentially responsible for the fluorescence remains unchanged. In such derivatives, basically similar fluorescence properties are to be expected. Of course, the parameters of the fluorescence, such as the wavelength of the absorption or the emission, may change. Due to the fact that the pyridine ring is essentially responsible for the fluorescence in NAD/NADH, derivatives in which the pyridine ring is not modified are particularly suitable. Derivatives suitable for the present invention are described, for example, in WO 2007/012494 and US 2007/0026476. CarbaNAD, a derivative without glycosyl binding which was already described in 1988, is particularly suitable for use in the method according to the invention (J. T. Slama, Biochemistry 1989, 27, 183 and Biochemistry 1989, 28, 7688). The ribose is substituted therein by a carbocyclic sugar unit. Of course, not all derivatives of NADH/H⁺ and NADPH/H⁺ are equally suitable for the invention. The suitability for use according to the invention can, however, be experimentally examined without problems.

The enzyme of an enzyme/coenzyme pair used in the context of the invention is preferably selected from the group comprising glucose dehydrogenase (E.C.1.1.1.47), lactate dehydrogenase (E.C.1.1.1.27, 1.1.1.28), malate dehydrogenase (E.C.1.1.1.37), glycerin dehydrogenase (E.C.1.1.1.6), alcohol dehydrogenase (E.C.1.1.1.1), or amino acid dehydrogenase, e.g., L-amino acid dehydrogenase (E.C.1.4.1.5).

As described, the fluorescence lifetime correlates so well with the concentration of the analyte that it can be used, in the evaluation algorithm for calculating the analyte concentration, independently as the measurement value which is characteristic for the concentration. However, it is preferably used in combination with another measurement variable which characterizes the concentration, in particular the fluorescence intensity, or also the optical absorption. It can also be applied in order to compensate for measurement errors, which are caused by interfering variables, in particular the temperature of the sample or its hematocrit.

The features disclosed in the above description, the claims and the drawings may be important both individually and in any combination with one another for implementing the invention in its various embodiments.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Having described the present invention in detail and by reference to specific embodiments thereof, it will be apparent that modification and variations are possible without departing from the scope of the present invention defined in the appended claims. More specifically, although some aspects of the present invention are identified herein as preferred or particularly advantageous, it is contemplated that the present invention is not necessarily limited to these preferred aspects of the present invention.

What is claimed is:

1. A method for determining the concentration of glucose in a liquid sample using a reagent system which contains glucose dehydrogenase (GlucDH), comprising:
(a) providing an analysis element in which the reagent system is operatively integrated;
(b) reacting the sample with the reagent system in order to produce a change of the quantity of a fluorophore, the fluorophore being selected from the group comprising NADH/H⁺ and NADPH/H⁺, including their derivatives, wherein the quantity of the fluorophore correlates with the concentration of the analyte and wherein the reaction of the sample with the reagent system includes a complex formation with participation of the GlucDH and the fluorophore;
(c) measuring a measurement variable which is characteristic for the quantity of the fluorophore; and
(d) determining the concentration of the analyte on the basis of the measurement variable using an evaluation algorithm;
wherein fluorescence lifetime of the fluorophore is used in the determining the concentration of the analyte.

2. The method according to claim 1, wherein the fluorophore is a derivative of NADH/H⁺ or NADPH/H⁺, whose pyridine ring is not modified or is carbaNAD.

3. The method according to claim 1, wherein the fluorescence lifetime of the fluorophore is used in the evaluation algorithm as the measurement variable which is characteristic for the concentration.

4. The method according to claim 1, wherein the measured fluorescence lifetime is used in the evaluation algorithm in combination with an other measurement variable which is characteristic for the concentration, wherein the other measurement variable comprises fluorescence intensity.

5. The method according to claim 4, wherein the measured fluorescence lifetime is used in the evaluation algorithm to compensate for measurement errors which are caused by interfering variables.

6. The method according to claim 1, wherein the measurement of the fluorescence lifetime is used to determine a lifetime-related value of the fluorescence intensity.

7. The method according to claim 6, wherein the fluorescence intensity is measured for at least two different fluorescence lifetimes in order to determine in each case lifetime-related values of the fluorescence intensity.

8. The method according to claim 7, wherein the two lifetime-related values of the fluorescence intensity are related to one another.

9. An analysis system for determining the concentration of an analyte in a liquid sample, the system comprising a reagent system containing glucose dehydrogenase (GlucDH), whose reaction with the sample results in a change of the quantity of a fluorophore, the fluorophore comprising one of NADH/H⁺ and NADPH/H⁺ including their derivatives, the quantity of the fluorophore correlating with the concentration of the analyte, the reaction of the sample with the reagent system including a complex formation with participation of the GlucDH and the fluorophore, and an evaluation instrument comprising a measuring unit for measuring a measurement variable which is characteristic for the quantity of the fluorophore, and an evaluation unit for determining the concentration of the analyte on the basis of the measurement variable, using an evaluation algorithm, wherein the reagent system comprises reagents contained in dry form in an analysis element, and wherein the measuring unit of the evaluation instrument is adapted for measuring and/or taking into account the fluorescence lifetime of the fluorophore.
